SEQUENCE LISTING (31 sequences)

SEQ ID NO: 1 - DNA, 2475 bp, Pyrus communis
  CDS (1)..(2091)
tgggagttggaattcaaacaatgtggagcattctgctattgttttcctgcattttttctgcaagcttcggcttctgtgagttacgaccacaaggctataataattaatgggcagaaaaggattttaatttctggctccattcactatcccagaagcactcctgagatgtggccggatttaattcagaaggccaaagatggaggcttggatgttatacagacctatgtgttttggaatggccacgaaccttctccgggaaaatattatttcgaggacagatatgatttggtcaagttcatcaagctggtgcaacaagcaggcctatttgttaatctccggattggcccttatgtttgcgctgaatggaacttcgggggattcccagtttggctgaaatatgtccctggaatcgcttttcgaacggacaatgagcctttcaaggcggcaatgcaaaaatttacagagaagattgtcagcatgatgaaggcagagaagctgtttcaaagtcaaggaggtcctataattctctctcagatagaaaatgaatttggacctgtggaatgggaaattggtgcacctggaaaagcttacaccaaatgggcagctcagatggctgtaggtctagacactggagttccatggattatgtgcaagcaagaggatgcccccgatcccgttattgacacttgcaatggtttctactgtgagaatttcaagccaaataaggactataagcccaaaatgtggacagaagtctggactggttggtatacagaattcggtggggcagttcccactagacctgcagaagatgtggcattttcagttgctaggttcatacaaagcggcggttcgtttttgaactattacatgtaccacggaggaacgaattttggccgaacagccggaggtcccttcatggccactagctatgactatgacgcccccttagacgaatatggactaccccgggaaccaaagtggggacatttgagagatctgcacaaagccattaaaccatgtgagtctgctttagtgtccgttgatccttcagtgactaaactcggaagtaatcaagaggctcatgtattcaaatcagagtcggattgcgctgcattcctcgcaaattatgacgcaaaatactctgttaaagtgagctttggaggcgggcagtatgacctgccgccatggtccatcagcattcttccggactgcaaaaccgaagtttacaacactgcaaaggttggttcgcaaagctcgcaagttcagatgacaccagtacatagtggatttccttggcagtcattcatcgaagaaaccacctcttctgatgagaccgatacaacttacatggacgggttgtatgagcaaataaatatcactagggatactacagactacttgtggtacatgacagatatcacaataggttctgatgaagcatttctaaagaacggaaagtccccgcttcttacaatctcttcagcaggtcatgccttgaatgttttcatcaatggtcagctctcaggaaccgtgtatgggtcgttggagaatcctaaattatcattcagtcaaaacgtgaacctgagatctggcatcaacaagcttgcattgcttagcatttccgttggtctgccgaatgttggtactcactttgagacatggaacgcgggagttcttggcccgatcacattgaaaggtctgaattcaggaacatgggacatgtcagggtggaaatggacatacaagactggtctgaaaggtgaagctttaggcctccatactgttactgggagttcttctgttgaatgggtagaaggaccatcgatggctaaaaaacaaccccttacgtggcacaaggctacttttaatgcaccaccaggtgatgctccattagctttagatatgggaagcatgggaaaaggtcagatatggataaatggacagagcgtggacgccactggcctggatacattgcacgcggcagctgtggcgattgttcttatgccggaacttatgatgataagaaatgcagaactcattgcggcgagccctctcagagatggtaccacattcctcgatcgtggttgaccccgactggaaatcttttggtggtgttcgaagaatggggcggtgatccgtcagggatttcgttggttgaaagaggtacagccctcgacgcgaagaagctctaggttgaggctgtctgcagctaaagatcgagcagatacgtagattactaaatacgtgaagtggttgtgtacatagacaatctattaattgtcgaaaaaaaatatagctccacatgatatacgaagggttacatacaaagtttgtagtcagtagatttgcgcaagcattttccattgtaagtttgtaacaacttatggaaaagatttccttttcctttacaagaataaatggaaaactaatagagactactttatccttgtctttctaaaaaaaaaaaaaaaa

SEQ ID NO: 2 - protein, 696 aa, Pyrus communis
WELEFKQCGAFCYCFPAFFLQASASVSYDHKAIIINGQKRILISGSIHYPRSTPEMWPDLIQKAKDGGLDVIQTYVFWNGHEPSPGKYYFEDRYDLVKFIKLVQQAGLFVNLRIGPYVCAEWNFGGFPVWLKYVPGIAFRTDNEPFKAAMQKFTEKIVSMMKAEKLFQSQGGPIILSQIENEFGPVEWEIGAPGKAYTKWAAQMAVGLDTGVPWIMCKQEDAPDPVIDTCNGFYCENFKPNKDYKPKMWTEVWTGWYTEFGGAVPTRPAEDVAFSVARFIQSGGSFLNYYMYHGGTNFGRTAGGPFMATSYDYDAPLDEYGLPREPKWGHLRDLHKAIKPCESALVSVDPSVTKLGSNQEAHVFKSESDCAAFLANYDAKYSVKVSFGGGQYDLPPWSISILPDCKTEVYNTAKVGSQSSQVQMTPVHSGFPWQSFIEETTSSDETDTTYMDGLYEQINITRDTTDYLWYMTDITIGSDEAFLKNGKSPLLTISSAGHALNVFINGQLSGTVYGSLENPKLSFSQNVNLRSGINKLALLSISVGLPNVGTHFETWNAGVLGPITLKGLNSGTWDMSGWKWTYKTGLKGEALGLHTVTGSSSVEWVEGPSMAKKQPLTWHKATFNAPPGDAPLALDMGSMGKGQIWINGQSVDATGLDTLHAAAVAIVLMPELMMIRNAELIAASPLRDGTTFLDRG

SEQ ID NO: 3 - DNA, 1673 bp, Pyrus communis
  CDS (112)..(1308)
cctttcaactactcgttctaaagtaattaagacaagtagcctctttatttctcctacatctccatctcactcctttttcaaatcagaaaatcctaaaaccagccagcacaaatggcaaaccccaaaagcctctcatatccagcagctgcagtttttgcgttgttgatgatggctataagcattactaatgtggatgctgcagccgtcactttcagtgtgagcagtttaggagccaaagcagatggcagtactgactccaccaaggccttcctctctgcgtggtccaatgcttgtgcctccgtcaaccctgctgtcatatatgtccccgcagggaggttcttgcttggcaatgccgtgttctctgggccatgcaagaacaacgccatcaccttccgcattgccggcactctcgtcgccccgtctgattaccgggtcattggaaatgccggtaactggcttctctttcagcatgtcaatggggtcacgatttccggtggagttctcgacggtcagggcaccggattgtgggattgcaagtcctcgggcaagagttgccccagcggagcaactacactgagcttttcgaactccaacaacgttgtggtgagtggattaatatcactaaacagccaaatgttccacattgtcgtcaacggctgccaaaatgtgaaaatgcaaggtgtcaaggttaacgcggccggcaacagccccaacaccgatggcatccatgtccaaatgtcatctggagtcaccattctcgactccaaaatttcaaccggtgacgactgtgtctcagttggccccggcactaccaatttgtggattgaaaacgtcgcatgtggacccggccacggaatcagcattgggagtttagggaaggaccaacaagaagccggtgtacaaaatgttacagttaaaacagttacattcactggtactgaaaacggcgtcagaattaagtcttgggggagacctagcactggatttgctaggagcattcttttccaacatattgtgatgaccaacgttcaaaatccaatcgttattgatcaaaattactgccctaatgacaaaggttgccctggccaagcttctggagttaaggtcagcgatgtgacgtatcaagacattcatggtacatcggcgacggaagtggcggtgaaattcgattgtagttccatgtatccttgcaacgggatcagactgcaagatgtgaagctcacttacaataaccaagcagctgaagcttcctgcatccatgcaggcggaacaactgccggtacggttcagccgacaagttgtttctaactcgagttgtagttttttccatctactcctcctcactcggagtctcgtagtactagttgggataaaaaagaagggactagtcatactataaactatatatatatatatatatataagaattaaagaatatttctagagtagtaggtctaggtctagctctagctctacgtagttgatgtattgagatgtattttgcttgagcctgccgtgttggcagcctattgggcttccttagagcctggcgctgcatcatccaaacccacttcatggagagattctcttttgcattgggtgctttgtattatggaatgttgtaacttgaaagtgataaatgcaatatgaattaaaagtaaaaaaaaaaaaaa

SEQ ID NO: 4 - protein, 398 aa, Pyrus communis
MANPKSLSYPAAAVFALLMMAISITNVDAAAVTFSVSSLGAKADGSTDSTKAFLSAWSNACASVNPAVIYVPAGRFLLGNAVFSGPCKNNAITFRIAGTLVAPSDYRVIGNAGNWLLFQHVNGVTISGGVLDGQGTGLWDCKSSGKSCPSGATTLSFSNSNNVVVSGLISLNSQMFHIVVNGCQNVKMQGVKVNAAGNSPNTDGIHVQMSSGVTILDSKISTGDDCVSVGPGTTNLWIENVACGPGHGISIGSLGKDQQEAGVQNVTVKTVTFTGTENGVRIKSWGRPSTGFARSILFQHIVMTNVQNPIVIDQNYCPNDKGCPGQASGVKVSDVTYQDIHGTSATEVAVKFDCSSMYPCNGIRLQDVKLTYNNQAAEASCIHAGGTTAGTVQPTSCF

SEQ ID NO: 5 - DNA, 700 bp, Pyrus communis
  CDS (1)..(588)
gaattgggcccgacgtcgcatgctcccggccgccatggccgcgggattgcagtggtggcaaaagatggaacgggaaactttcagacggtgaaagaggccatggatgcggctgatgggaaaaaaaggtttgtgatttacgtgaaagcaggagtttataaggagaaaattcacagtaataaagacgggattactttgatcggagatggtaaatattccaccatcattgtcggtgatgatagtgttgctggaggttccaccatgccaggctctgcaactattacaatgacaggggatggattcatagcccgcgacattgggtttcagaacacagcagggccacaaggagagcaagctttagctctaaacatagcttctgatcactctgttctttacaggtgcagcattgcgggttaccaggatactctctacgcacacgctctccgtcaattctacagagaatgcgacatctacggcaccgtcgatttcattttcggaaacgccgccgcggttttccaaaactgctacttggttcttcgtcttcctcggaaaaaaggctacaacgttattctaaaaaacggaagatcctgacccgggacagaacactgggtttctctgttcacaacttgcagaatcgtacccagctccgaattttctccggtaaaacataagtaccgaatcgtatcttggtaggccatggaaa

SEQ ID NO: 6 - protein, 195 aa, Pyrus communis
ELGPTSHAPGRHGRGIAVVAKDGTGNFQTVKEAMDAADGKKRFVIYVKAGVYKEKIHSNKDGITLIGDGKYSTIIVGDDSVAGGSTMPGSATITMTGDGFIARDIGFQNTAGPQGEQALALNIASDHSVLYRCSIAGYQDTLYAHALRQFYRECDIYGTVDFIFGNAAAVFQNCYLVLRLPRKKGYNVILKNGRS

SEQ ID NO: 7 - DNA, 1276 bp, Pyrus communis
  CDS (65)..(841)
caatcctattcatcactctctctcctctctctgctttctcactcccctttctttctctccggcaatggcctcccttcgcgtcctctacattgctttcatgctctcactcttcatggaggccaacgctagaattccaggagtttacactggtggcccatgggagggcgcccacgccaccttctacggtggcaacgacgcctctggaaccatgggtggcgcttgcgggtacggaaacctctacagccaaggctacggcgtgaacacggcggcactgagcactgctctgttcaacaatggccttagctgcggcgcctgcttcgagattaagtgcggcgacgaccccaggtggtgccacccaggcaacccctccatcttagtcaccgccaccaacttctgccctcctaacttcgctcagcccagcgacgacggcgggtggtgcaaccctccccgcacccatttcgacctcgccatgcccatgttcctcaagatcgccgagtacaaggccggcatcgtccccgtctcttaccgccgagttccgtgcagaaagcaaggcggagtgagattcacaattaacggtttccgttacttcaacctggttctgatcaccaacgtcgcgggcgcaggggatatcgtgagggtgagcgtaaaaggcgcgaacactggatggatgccgatgagccgcaactggggacaaaactggcaatccaacgcagacctggtgggccagaccctgtcgtttcgagtcacgggcagtgacaggcgcacatccacctcccacaacgtggcacccgctgattggcagttcggacaaactttcaccggcaagaatttccgggtctaaaattaagaagggaaaaaaaagtttatccactatctttaattttccttttgggtttttaactttttttttaaattatcaaagtttaatttcccgccatctgattttccttaattttcccgggaaaatttggaagcggtgggagtataaaagtaaaagtattagatgatgtggggttaaaagttaaaattgggtggtaagataggtcgaaaagcgacttcttttgcaagtgtggtgtgcggcaactttttacttttggtgcttttttttttaggtttgagtgggaggctggtaaaaatttaggtgatccggccaaatagtgcgtgtaaaaggagttgaagcggctgcaaataaccaacgtgcagcccgcagctctacccatatcttttctagaattttatatgatatatatttcagttagcaaaaaaaaaaaaaaaa

SEQ ID NO: 8 - protein, 258 aa, Pyrus communis
MASLRVLYIAFMLSLFMEANARIPGVYTGGPWEGAHATFYGGNDASGTMGGACGYGNLYSQGYGVNTAALSTALFNNGLSCGACFEIKCGDDPRWCHPGNPSILVTATNFCPPNFAQPSDDGGWCNPPRTHFDLAMPMFLKIAEYKAGIVPVSYRRVPCRKQGGVRFTINGFRYFNLVLITNVAGAGDIVRVSVKGANTGWMPMSRNWGQNWQSNADLVGQTLSFRVTGSDRRTSTSHNVAPADWQFGQTFTGKNFRV

SEQ ID NO: 9 - DNA, 1144 bp, Pyrus communis
  CDS (83)..(850)
actccacctgccctacacaaaaactaaaactcctctctttcttttccctattgaaatcaaaacccaccaaaaagccacaaaaatggcagctcatgcattgtcttttgctcctatagccctctctgttgttctctttaatctacatctgcatggtgtatttgctgtttatggtagctgggaaggcgctcatgccacattttacggtggcggtgatgcttctggcacaatgggaggagcatgtggttatgggaatttgtacagccaggggtatggaaccaacactgcagctttgagcacaagcattgttcaacaatggcttaagctgtgggtcttgttatgaaatgagatgcgacaatgacccgagatggtgccgtcctggatccatcattgtaactgctacaaacttttgccctcctaactttgctcagtccaacgacaatggcggatggtgcaatcctcctctccagcatttcgatttggctgagcctgctttcttgcaaattgcccaataccagtgctggaatcagtgccaggtttccttcagaagagtaccttgtgtgaagaaaggaggaataagattcaccatcaacggccactcctacttcaacctggttttgatcaccaacgtggctggggcaggagacgtccattcagtttcaatcaaggggtccagaacagggtggcaacccatgtcaagaaactggggtcaaaactggcagagcaactcttacctcaatggccaagccctctccttccaagtcaccaccagtgacggtagaaccgtcacgagctacaacgtcgcgcctggtaattggcagtttggtcagacattctccgggggtcaactttagagatattcctctacattattggtaaaaatttgtatatctatctgtcatttttttcccgtaaacttttttgagtgtaaaagcaaagagtagttgtgaagtggaggtttgctgaggtgagctaaaaaaacacccgctgggcctttcacatttgagttttcctggagaaatgatattcacctcattcaggttgtaaccaatttctcagttgtacttgtaaccttaatgatatatatatttataaaaaacgagaaagctttatcaagtaaaaaaaaaaaaaagaaaaaaaaaaaaaa

SEQ ID NO: 10 - protein, 85 aa, Pyrus communis
MAAHALSFAPIALSVVLFNLHLHGVFAVYGSWEGAHATFYGGGDASGTMGGACGYGNLYSQGYGTNTAALSTSIVQQWLKLWVLL

SEQ ID NO: 11 - protein, 5 aa, Pyrus communis
NEMRQ

SEQ ID NO: 12 - protein, 19 aa, Pyrus communis
PEMVPSWIHHCNCYKLLPS

SEQ ID NO: 13 - protein, 21 aa, Pyrus communis
LCSVQRQWRMVQSSSPAFRFG

SEQ ID NO: 14 - protein, 95 aa, Pyrus communis
ACFLANCPIPVLESVPGFLQKSTLCEERRNKIHHQRPLLLQPGFDHQRGWGRRRPFSFNQGVQNRVATHVKKLGSKLAEQLLPQWPSPLLPSHHQ

SEQ ID NO: 15 - protein, 10 aa, Pyrus communis
NRHELQRRAW

SEQ ID NO: 16 - protein, 13 aa, Pyrus communis
LAVWSDILRGSTL

SEQ ID NO: 17 - DNA, 24 bp, artificial sequence
  Degenerated primer
tggytcyattcaytayccyagaag

SEQ ID NO: 18 - DNA, 24 bp, artificial sequence
  Degenerated primer
cahganckwggaayrtgrtaccat

SEQ ID NO: 19 - DNA, 24 bp, artificial sequence
  designed specific primer
gcctccatctttggccttctgaat

SEQ ID NO: 20 - DNA, 23 bp, artificial  sequence
  misc_feature (1)..(23): Ionosine
agyccyaayacygayggnatyca

SEQ ID NO: 21 - DNA, 23 bp, artificial sequence
  degenerated primer
arrctnccratrctkatnccrtg

SEQ ID NO: 22 - DNA, 23 bp, artificial sequence
  specific primer
agtcgagaatggtgactccagat

SEQ ID NO: 23 - DNA, 23 bp, artificial sequence
  specific primer
ggcactaccaatttgtggattga

SEQ ID NO: 24 - DNA, 21 bp, artificial sequence
  specific primer
accgtcgatttcattttcgga

SEQ ID NO: 25 - DNA, 21 bp, artificial sequence
  specific primer
aaaccatggcctaccaagata

SEQ ID NO: 26 - DNA, 21 bp, artificial sequence
  specific primer
ccctgtattgtaatagttgca

SEQ ID NO: 27 - DNA, 22 bp, artificial sequence
  degenerated primer
acrwyggyggntggtgyaaycc

SEQ ID NO: 28 - DNA, 21 bp, artificial sequence
  degenerated primer
tgccarttkksncccarttyc

SEQ ID NO: 29 - DNA, 23 bp, artificial sequence
  specific primer
cggtattgggcaatttgcaagaa

SEQ ID NO: 30 - DNA, 23 bp, artificial sequence
  specific primer
ggatatcgtgagggtgagcgtaa

SEQ ID NO: 31 - DNA, 23 bp, artificial sequence
  specific primer
ggagacgtccattcagtttcaat

(19) United States
(12) Patent Application Publication
(10) Pub. No.: US 2004/0049809 A1
(43) Pub. Date: Mar. 11, 2004

(54) Pear genes codifying for beta-galactosidase,pectin methylesterse, polygalacturonase, expansins and their use

(76) Inventors: Sandra Cristina Matias Fonseca, Loures (PT); Aladje Balde, Queluz (PT); Maria Salome Soares Pais, Lisboa (PT)

Correspondence Address: FINNEGAN, HENDERSON, FARABOW, GARRETT & DUNNER, LLP, 1300 I STREET, NW, WASHINGTON, DC, 20005, US

(21) Appl. No.: 10/362,091

(22) PCT Filed: Aug. 20, 2001
(86) PCT No.: PCT/PT01/00021

(30) Foreign Application Priority Data
Aug. 22, 2000 (PT) 102511 C

(51) Int. Cl. A01H 1/00; C12N 15/82; C07H 21/04; C12N 9/16; C12N 9/24; C12N 9/40; C12N 5/04

(52) U.S. Cl. 800/284; 435/69.1; 435/196; 435/208; 435/200; 435/320.1; 435/419; 536/23.2

(57) ABSTRACT

This invention provides isolated and purified nucleotide sequences which are differentially expressed during pear fruit ripening, and their protein products. The isolated genes can be inserted into expresssion cassettes and cloned in an expression vector which can be used to transform a host cell by selected transformation methods. Transgenic plants can be regenerated from transformed plant cells by in vitro culture techniques. The nucleotide sequences disclosed in this invention encode proteins which are described as having an effective action in fruit ripening control. When used in antisense orientation they can delay fruit softening and mesocarp deterioration, bringing important advantages for fruit producers.

### FIELD OF THE INVENTION

The present invention relates to the isolation and identification of nucleotide sequences encoding for proteins involved in ripening pear fruits, a method for regulating fruit ripening by transforming plants with a construct containing one or more of the isolated genes, and transgenic plants and seeds transformed with such constructs.

### BACKGROUND OF THE INVENTION

Pears are the third most important fruit produced in temperate regions after grapes and apples.

Pear (Pyrus communes L.) epidermis is very sensitive to transport and handling, small mechanical shocks give rise to mesocarp deterioration and precocious pear senescence. Pears are harvested at commercial maturity (a full growing green stage) and cold stored. The onset of ripening starts when the fruits leave the cold, and it takes only two weeks until the fruit reaches an overripe phase. This means that most of the time when pear fruits reach the consumers they are overripen. To avoid this, the producers have to harvest pears before they reach the optimal maturation stage. Often these fruits fail to ripen with full organoleptic quality. This constitutes a problem for fruit producers, which has considerable losses in fruit flowing off, and for consumer, which often buy a fruit with poor quality. For all that we can understand why only about 10% of the pears produced in Portugal, for example, are exported (Azevedo, 1997, Revista do Agricultor 104/105:45-48).

At the present time producers have the need to control pear fruit ripening so they started to test the application of chemical products to delay fruit ripening. The molecular approach described in this patent provides the ripening control by antisense expression of ripening related genes without use of chemical substances and with no changes in the organoleptic characteristics of such tasty fruit.

Extensive cell wall modifications that occur during fruit ripening are thought to underlie processes such as fruit softening, tissue deterioration, and pathogen susceptibility. These modifications are regulated at least in part by the expression of genes that encode cell wall-modifying enzymes (Fisher and Bennett, 1991, Annu. Rev. Plant Physiol. Plant Mol. Biol., 42:675-703). Pectins are a major class of cell wall polysaccharides that are degraded during ripening, undergoing both solubilization and depolymerization. In tomato the majority of ripening-associated pectin degradation is attributable to the cell wall hydrolase Polygalacturonase (Hadfield et al., 1998, Plant Physiol., 117:363-373).

Polygalacturonase (PG) catalyze the hydrolytic cleavage of α-(1→4) galacturonan linkages of pectic backbone (Fisher and Bennett, 1991, Annu. Rev. Plant Physiol. Plant Mol. Biol., 42:675-703). PG has been extensively studied in tomato fruit, where it accumulates during ripening and is responsible for the degradation of polyuronides in fruit cell wall (Smith et al., 1988, Nature, 334:724-726). However, experiments using transgenic tomato plants with altered PG gene expression indicated that PG-dependent pectin degradation is neither required nor sufficient for tomato fruit softening to occur (Sheehy et al., 1988, Proc. Natl. Acad. Sci. USA, 85:8805-8809; Smith et al., 1988, Nature, 334:724-726; Giovannonni et al., 1989, Plant Cell, 1:53-63). Data from experiments using fruit of the same transgenic lines strongly suggested that PG-mediated pectin degradation is important in the later, deteriorative stages of ripening and in pathogen susceptibility of tomato fruit (Schuch et al., 1991, Hortscience, 26-:1517-1520; Kramer et al., 1992, Post. Biol. Tech., 1:241-255; Hadfield et al., 1998, Plant Physiol., 117:363-373).

Polygalacturonase is known to be more active in degrading demethylated than methylated pectin (Fisher and Bennett, 1991, Annu. Rev. Plant Physiol. Plant Mol. Biol., 42:675-703). Pectin methylesterase (PME) is a cell wall metabolizing enzyme responsible for the demethylation/de-esterification of galacturonic acid residues in high molecular weight pectin (Hall et al., 1993, The Plant J., 3(1): 121-129). In tomato, PME is present throughout fruit development with activity increasing two to three-fold during ripening (Hobson, 1963, Biochem. J., 86:358-365; Harriman et al., 1991, Plant Physiol., 97:80-87). As the methylesterification level (60%) seems to protect the homogalacturonans (HGA) from a more extended PG activity, it has been thought that PME play an important role in the determination of the extension in which the pectins are susceptible to PG action (Dick and Labavitch, 1989, Plant Physiol., 89:1394-1400). Inhibition of fruit-specific PME gene expression by its antisense gene, in tomato, results in loss of tissue integrity of fruit pericarp but does not affect the growth and development of tomato plant (Tieman et al., 1992, Plant Cell, 4:667-679; Hall et al., 1993, The Plant J. 3(1): 121-129; Tieman and Handa, 1994, Plant Physiol., 106:429-436).

Although some loss of galactosyl residues could result indirectly from the action of PG, β-Galactosidase (β-Gal) is the only enzyme identified in higher plants capable of directly cleaving β-(1,4) galactan bonds, and probably plays a role in galactan side chain loss (De Veau et al., 1993, Physiol. Plantarum, 87:279-285; Carey et al., 1995, Plant Physiol., 108:1099-1107; Carrington and Pressey, 1996, J. Am. Soc. Hortic. Sci., 121:132-136; Smith et al., 1998, Plant Physiol., 117:417-423). Studies in apple, melon, kiwi and avocado (Ranwala et al., 1992, Plant Physiol., 100:1318-1325; Ross et al., 1993, Planta, 189:499-506; Ross et al., 1994, Plant Physiol., 106:521-52.8) suggests that β-Gal acts like a galactanase hidrolyzing the neutral sugar polimers which attach the ramnogalacturonan backbone from pectins to the hemicelluloses (Lazan et al., 1995, Physiol. Plantarum, 95:106-112; Ranwala et al., 1992, Plant Physiol., 100:1318-1325). Several studies suggest that β-gal can significantly contribute to pectin and hemicellulose modification, assuming an especially important role in the later stages of fruit ripening. That activity could be complemented by PG, cellulases and other glycosidases action (Carey et al., 1995, Plant Physiol., 108:1099-1107).

Unlike the enzymes described above, Expansins lack hydrolytic activity (McQueen-Mason et al., 1992, Plant Cell, 4:1425-1433; McQueen-Mason et al., 1993, Planta, 190:327-331). Instead, Expansins appear to disrupt the noncovalent bonding between cellulose and hemicellulose, thereby allowing the wall polymers to yield to the turgor-generated stresses in the cell wall (Cosgrove, 1997, Proc. Natl. Acad. Sci. USA, 94:5504-5505). This results in a relaxation of wall stress and turgor pressure and, consequently, an uptake of water to enlarge the cell and expand the wall (Cosgrove, 1993, New Phytol., 124:1-23; Scherban et al., 1995, Proc. Natl. Acad. Sci. USA. 92:9245-9249). Expansin protein motifs are very conserved, however they play a role in different processes of cellular growth. An expansin gene from tomato was recently isolated and showed to be specifically and abundantly expressed in ripening fruit, when growth ceased and a strong cell wall degradation occurs (Rose et al., 1997, Proc. Natl. Acad. Sci. USA, 94:5955-5960; Rose et al., 2000, Plant Physiol., 123:1583-1592). Homolog cDNAs have already been isolated from other rapid ripening fruits like melon and strawberry. It is known that expansin expression is ethylene regulated which makes us to assume these proteins can also contribute to cell wall degradation in non-growing tissues, allowing a more efficient action of other endogenous enzymes on non-covalently linked polymers (Rose et al., 1997, Proc. Natl. Acad. Sci. USA, 94:5955-5960).

### SUMMARY OF THE INVENTION

Genes codifying for β-Galactosidase, Pectin Methylesterase, Polygalacturonase and two Expansin proteins were isolated from pear fruit. These enzymes are expressed during fruit maturation and ripening and can be used as targets for the generation of transgenic plants. The isolated genes can regulate the referred enzyme expression and thereby control aspects of plant development, and in particular fruit ripening.

These genes can be inserted in sense or antisense in pear and in other fruit species allowing the ripening control. By “antisense downregulation” and “sense downregulation or “cossupression”, the expression of a target gene can be inhibited. As a consequence the fruits can be collected later on ripening, with better organoleptic quality and reduced losses in transportation and storage.

### DETAILED DESCRIPTION AND PREFERED EMBODIMENTS OF THE INVENTION

The present invention provides new isolated genes from pear fruit particularly produced during the ripening process. These genes encode for cell wall hydrolases—β-Galactosidase (β-Gal), Pectin Methylesterase (PME) and Polygalacturonase (PG)—and for a novel class of cell wall proteins—Expansins (Exp1 and Exp2).

Also provided for this invention, the claimed nucleic acid sequence can be used to suppress the expression of endogenous β-gal, PME, PG, Exp1, and Exp2 genes in any fruit or other plant organs, thus modifying the structure of the cell walls of the fruit or plant and providing for ripe yet firm fruit and vegetables. This suppression can be achieved by “sense downregulation” or “cossuppression” or by “antisense downregulation”. mRNA, RNA, cRNA, cDNA and DNA molecules inserted in sense or antisense orientation can serve this purpose.

Nucleic Acids Sequences Isolation from Plants

The genes of the present invention may be isolated from ripening fruits using different methods well known in the art. In particular two approaches can be used. One is the approach described here which consists on degenerated primers design from conserved portions of sequence alignments, using sequences from the same gene isolated from other species published in the database. The other approach can be the construction of a cDNA library and screening using heterologous probes.

The procedures for isolating the DNA, RNA or cDNA encoding a protein according to the present invention, subjecting it to partial digestion, isolating DNA fragments, ligating the fragments into a cloning vector, and transforming a host are well known in recombinant DNA technology. Accordingly, one of ordinary skill in the art can use or adapt the detailed protocols for such procedures as found in Sambrook et al. (1989), Molecular Cloning: A Laboratory Manual, 2nd. Ed., Cold Spring Harbor, or any other manual on recombinant DNA technology. Fragments of the genes of the present invention are also contempled by the present invention.

The designed degenerated primers can be used to obtain isoenzymes of the same gene in Pyrus species or to isolate the homologous gene from other different species by PCR and other in vitro amplification methods. The specific designed primers can be replaced by different ones in order to obtain slightly different fragments of the same nucleic acid sequence claimed here. For a general overview of PCR see PCR Protocols: A Guide to Methods and Applications (Innis, M., Gelfand, D., Sninsky, J., and White, T., eds.) Academic press, San Diego (1990).

Polynucleotides can also be synthesized by well-known techniques as described in the technical literature. Double stranded DNA fragments may then be obtained either by synthesizing the complementary strand and annealing the strands together under appropriate conditions, or by adding the complementary strand using DNA polymerase with an appropriate primer sequence.

Once one coding gene of the present invention has been isolated from species, it can serve as a hybridization probe to isolate corresponding genes from the other species by cross-hybridization under low or moderate stringency conditions. Used as heterologous probes, the isolated genes can be used for screening a cDNA library or a genomic library, from any species. Used as homologous probes, the isolated nucleic acid sequences can be used to screen a library constructed from any species of Pyrus genus.

Substitution of one or more codons coding for an amino acid having similar chemical properties to the original one can be made creating an analog-coding gene. An analog may be defined as a peptide or fragment which exhibits the biological activity of the proteins of the present invention, and which is differentially expressed during fruit ripening.

Use of Nucleic Acids of the Invention to Inhibit Gene Expression

According to the present invention, a DNA molecule may also be operably linked to a promoter capable of regulating the expression of the said DNA molecule, to form a chimeric gene. That chimeric gene can be introduced into a replicable expression vector, for using in transforming plants. The replicable expression vectors may also be used to obtain the polypeptides coded by the genes of the present invention by well-known methods in recombinant DNA technology. [S1]

Replicable expression vectors usually comprise a promoter (at least), a transcription enhancer fragment, a termination signal, a translation signal, or a combination of two or more of these elements operably linked in proper reading frame. Preferably the vector encodes also a selectable marker, for example, antibiotic resistance. Replicable expression vectors can be plasmids, cosmids, bacteriophages and viruses.

The isolated sequences can be used to prepare expression cassettes useful in a number of techniques. For example, these expression cassettes can be used to suppress endogenous Exp1 or Exp2 gene expression. Inhibiting expression can be useful, for instance, in suppressing the extension of plant cell walls and disassembly of cell wall components.

The nucleic acid segment to be introduced generally will be substantially identical to at least a portion of the endogenous gene or genes to be repressed. However the sequence does not need to be perfectly identical to inhibit expression.

Several methods can be used to inhibit gene expression in plants, using the antisense technology. A nucleic acid segment of the interest gene can be operably linked to a promoter (CaMV 35S promoter or to a fruit specific promoter, for example) such that the antisense strand of RNA will be transcribed. That expression cassette can be then used to transform plants were the antisense strand of RNA will be produced. In plant cells, it has been suggested that antisense RNA inhibits gene expression by preventing the accumulation of mRNA which encodes the enzyme of interest, see e.g., van der Krol et al., 1988, Gene, 72:45-50.

For antisense supression generally higher homology can be used to compensate for the use of a shorter sequence. Normally, a sequence about 30 or 40 nucleotides and about full-length nucleotides can be used, but sequences between 200 and 500 nucleotides are especially preferred.

Catalytic RNA molecules or ribozymes can also be used to inhibit expression of the claimed genes. It is possible to design ribozymes that specifically pair with virtually any target RNA and cleave the phosphodiester backbone at a specific location, thereby functionally inactivating the target RNA. The inclusion of rybozime sequences within antisense RNAs confers RNA activity upon them, thereby increasing the activity of the constructs.

Another method of suppression is sense suppression. Introduction of expression cassettes in which a nucleic acid or a nucleic acid fragment is positioned in the sense orientation in frame with the promoter has shown to be an effective mean to block the transcription of target endogenous genes. See as revision article Stam et al., 1997, Annals of Botany, 79:3-12.

When sense inhibition of expression is desired, the introduced sequence should contain at least a fragment of the coding sequence or an intron or untranslated sequences homologous to sequences present in the primary transcript of the endogenous sequence. The introduced sequence should be substantially identical to the endogenous sequence intended to be repressed. The minimal identity should be typically greater than about 65%, but identities comprised between 80 to 100% are preferred. As in antisense suppression a higher identity in a shorter than full-length sequence compensates for a longer, less identical sequence. Nucleic acid sequences about 30 or 40 nucleotides may be used, but sequences between 200 and 500 nucleotides are especially preferred.

Use of Nucleic Acids of the Invention to Enhance Gene Expression

In opposition to the inhibiting fruit softening process, the nucleotide sequences of the invention can be used to accelerate the cell wall disassembly. This can be accomplished by the overexpression of the isolated sequences.

Use of Nucleic Acids of the Invention to Produce Transgenic Plants

The nucleic acid sequences isolated in the present invention can be incorporated in an expression vector and thereby be introduced into a host cell. Accordingly, one skilled in the art can use the sequences to make a recombinant cell. Suitable host cells include, but are not limited to, bacteria, virus, yeast, mammalian cells, insect, plant, and the like. Preferably the host cells are either a bacterial cell or a plant cell.

The nucleotide sequences claimed in this invention can be inserted in an expression vector, which may be introduced into the genome of the desired plant host by a variety of conventional techniques. The constructions using the isolated genes can be introduced into a conventional Agrobacterium tumefaciens host vector. The virulence functions of the Agrobacterium host will direct the insertion of the construct and adjacent marker into the plant cell DNA when the bacteria infect the cell.

Alternatively, the DNA constructs can be directly introduced into the plant cell genomic DNA using techniques such as electroporation and microinjection in plant cell protoplasts. Ballistics methods, such as DNA particle bombardment allows the DNA to be introduced directly in plant tissue.

Transformed plant cells derived by any of the above transformation techniques can be cultured to generate a whole plant, which possesses the transformed genotype and thus the desired phenotype such as increased fruit firmness. Such regeneration techniques rely on the manipulation of certain nutrients and phytohormones in a culture medium containing an antibiotic, herbicide or other marker that has been introduced together with the nucleotide sequences of interest. Regeneration can also be obtained from different plant explants or embryos. For a general overview, see Plant Cell, Tissue and Organ Culture. Fundamental Methods (O. L. Gamborg and G. C. Philips, eds.) Springer-Verlag (1995). Plant tissues suitable for transformation include, but are not limited to, floral buds, leaf tissue, root tissue, meristems, zygotic and somatic embryos, anthers, microspores and megaspores.

The resulting transformed plant with the genes of this invention may have an over expression or silencing pattern of β-gal and/or PME and/or PG and/or Exp1 and/or Exp2 genes. These plant fruits may have an abnormal ripening behavior: slower pulp softening, later mesocarp deterioration, increased fruit shelf life after harvest and an enhanced resistance against pathogenic attack. That is an example, if the isolated nucleotide sequences were used aiming the corresponding enzyme downregulation.

Fruit ripening control can be achieved in the transformed plants with constructions containing the isolated cDNA sequences. Moreover, the alterations produced in fruit tissue at cell wall level can interfere with the response to pathogens attack, namely to fungal attack, delaying or decreasing the extension of pathogen infection.

The DNA molecules of the present invention may be used to transform any plant in which expression of the particular protein encoded by said DNA molecules is desired. The DNA molecules of the present invention can be used over a broad range of plants, namely species from genera such as Asparagus, Avena, Brassica, Citrus, Citrullus, Capsicum, Castanea, Cucurbita, Daucus, Fragaria, Glycine, Hordeum, Lactuca, Licopersicon, Malus, Manihot, Nicotiana, Oryza, Persea, Pisum, Pyrus, Prunus, Raphanus, Secale, Solanum, Sorghum, Triticum, Vitis, Vigna, and Zea. The β-gal, PME, PG, Exp1 and Exp2 genes are particularly useful in the production of transgenic plants of Pyrus genus. It has to be understood that is not an exclusive list, but merely suggestive of the wide range of applicability of the DNA molecules of the present invention.

Any skilled person will recognize that an enzymatic activity assay, immunoassay, western blotting and other detection assays can be used to detect at the protein level, the presence or absence of the proteins which the isolated sequences encode for. At DNA level, Southern blotting, northern blotting and PCR analyses can be performed in order to determine, the effective integration of the desired gene sequences in the plant DNA, and the efficient gene expression or silencing due to the introduced sequences.

Any skilled person will recognize that after an expression cassette being stably incorporated in transgenic plants and confirmed to be operable, it can be introduced into other plants by sexual crossing. A number of standard breeding techniques can be used, depending on the species to be crossed. Transgenic seeds and propagules (e.g., cuttings) can be obtained and when cultured produce transgenic plants.

The embodiments described above and the following examples are provided to better illustrate the practice of the present invention and should not be used to limit the scope of the invention. It is understood that the invention is not restricted to the particular material, combinations of material, and procedures selected for that purpose. Numerous variations of such details can be implied and will be appreciated by those skilled in the art.

### EXAMPLES

### Example 1

Amplification of a β-Galactosidase Gene From Pear (Pcβgal)

Rocha Pear (Pyrus communis L. cv. Rocha) fruit mesocarp at different maturation stages was frozen in liquid nitrogen, grounded to a fine powder in a mortar and stored at −80° C. About 6 g of powder were mix with 20 ml of RNA extraction buffer for RNA extraction according the hot borate protocol (Wan and Wilkins, 1994, Anal. Biochem., 223:7-12). Messenger RNA (mRNA) isolation was performed with the Poly A Ttract System (Promega) according to manufacturer instructions. The RNA and mRNA pellet was stored in DEPC treated water at −80° C. Spectrophotometric quantification was performed in TE buffer. RNA and mRNA were electrophoresed on a 0.8% agarose gel at 80 V for 1.5 hr to check its integrity.

For the reverse transcription reaction (RT), 1 μg of pear mRNA and 25 U of Avian Myeloblastosis Virus (AMV) reverse transcriptase in a reaction mixture of 50 mM Tris-HCl pH 8.5, 8 mM MgCl2, 30 mM KCl and 1 mM DTT, containing 1.0 mM each dNTP, 12.5 μg BSA, 1.25 μg actinomicin D and 10 μM of oligo (dT) 17 (provided with 5′/3′ Race kit, Boehringer) was incubated for 90 min at 55° C. The cDNA produced was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of each degenerated primers BG1 (SEQ. ID. NO: 17) and BG2 (SEQ. ID. NO: 18). After an initial 5 min denaturation period at 94° C., the PCR parameters were 30 sec template denaturation at 94° C., 45 sec primer annealing at 45° C. and 2 min primer extension at 72° C. for 35 cycles. A final extension step of 10 min at 72° C. was used subsequently to ensure full-length amplification products. The termocycler used was a Perkin Elmer—Gene Amp PCR System 2400.

The obtained products were purified from the agarose gel and ligated into the vector pBluescript (KS+) (Stratagene). The ligated mixture was used to transform E. coli DH5α. Transformants were selected on LB agar plates containing ampicilin (100 μg/ml) X-gal (80 μg/ml) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The two bands obtained by PCR have approximately 2.0 and 2.3 Kb. The nucleotide sequences were sent to NCBI data bank that has shown significant homology with β-galactosidases isolated from other species. Both obtained bands correspond to the same gene sequence resulting, the smaller one from amplification with BG1 (SEQ. ID. NO: 17) and BG2 (SEQ. ID. NO: 18) primers, and the larger one from BG1 (SEQ. ID. NO: 17) and oligo (dT) 17 primer (Boehringer) (which has been used in the RT reaction). As the obtained sequence corresponds to about 90% of the gene coding region, a new specific antisense primer BG3 (SEQ. ID. NO: 19) (see Table 1) was designed to perform 5′ RACE (Rapid Amplification of cDNA Ends) reaction.

In order to perform 5′ RACE reactions, Marathon kit (Clontech) cDNA synthesis reaction was done using 4 μg of pear mRNA. The adapter ligation allows the use of AP1 (Adaptor Primer, provided with Marathon kit, Clontech) primer in amplification reaction. Marathon cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of primers BG3 (SEQ. ID. NO: 19) (see Table1) and AP1 (Clontech). After an initial 5 min denaturation period at 94° C., the PCR parameters were 30 sec at 94° C., 45 sec at 60° C. and 45 sec at 72° C. for 35 cycles and a final extension step of 10 min at 72° C. The 150 bp PCR product was cloned and sequenced as described above.

Fused together the 2.3 Kb sequence and the 0.150 Kb sequence represented about 95% of the complete coding region for pear β-galactosidase protein.

The β-galactosidase nucleotide sequences (SEQ. ID. NO: 1 ) was sent to NCBI data bank and has shown significant homology with β-galactosidases isolated from other species. The highest homology found at the DNA level using the blastn program was 96% with Pyrus pyrifolia mRNA clone # AB046543. Searches in all the available protein and DNA data banks failed to find 100% homology with any existing clone.

### Example 2

Amplification of a Polygalacturonase Gene From Pear (PcPG)

Pear mesocarp processing, RNA extraction, mRNA isolation and RT reaction were performed exactly as described for β-galactosidase isolation in Example 1.

The cDNA produced was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of each degenerated primers PG1 (SEQ. ID. NO: 20) and PG2 (SEQ. ID. NO: 21) (see Table1). After an initial 5 min denaturation period at 94° C., the PCR parameters were 30 sec template denaturation at 94° C., 30 sec primer annealing at 55° C. and 45 sec primer extension at 72° C. for 35 cycles. A final extension step of 10 min at 72° C. was used subsequently to ensure full-length amplification products. The termocycler used was a Perkin Elmer—Gene Amp PCR System 2400.

The obtained product was purified from the agarose gel and ligated into the vector pBluescript (KS+) (Stratagene). The ligated mixture was used to transform E. coli DH5α. Transformants were selected on LB agar plates containing ampicilin (100 μg/ml) X-gal (80 μg/ml) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The PCR obtained band has approximately 160 bp that corresponds only to 10% of coding region. In order to isolate whole gene RACE reactions were performed—5′ RACE reaction using the Marathon cDNA and 3′ RACE using cDNA from an RT performed as described in Example 1. Also, new primers were designed: PG3 (an antisense primer for 5′ RACE) (SEQ. ID. NO: 22) and PG4 (a sense primer for 3′ RACE) (SEQ. ID. NO: 23).

For 5′ RACE reaction, Marathon cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of primers PG3 (SEQ. ID. NO: 22) (see Table 1) and AP1 (provided with Marathon kit, Clontech). After an initial 5 min denaturation period at 94° C., the PCR parameters were 30 sec at 94° C., 45 sec at 52° C. and 1 min 20 sec at 72 ° C. for 35 cycles and a final extension step of 10 min at 72° C. The approximately 700 bp PCR product was cloned and sequenced as described above.

For the 3′ RACE reaction cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of primers PG4 (SEQ. ID. NO: 23) (see Table1) and Vial9 primer (provided with 5′/3′ Race kit, Boehringer). After an initial 5 min denaturation period at 94° C., the PCR parameters were 30 sec at 94° C., 45 sec at 45° C. and 2 min at 72° C. for 35 cycles and a final extension step of 10 min at 72° C. The approximately 800 bp PCR product was cloned and sequenced as described for the 160 bp fragment.

All the three isolated polygalacturonase fragments together comprise a cDNA molecule of 1673 bp in size (SEQ. ID. NO: 3) and represent 100% of the coding region. The complete nucleotide sequence was sent to NCBI data bank and has shown significant homology with polygalacturonases isolated from other species. The highest homology found at the DNA level using the blastn program was 81% with Prunus persica mRNA clone # AF095577. Searches in all the available protein and DNA data banks failed to find 100% homology with any existing clone.

### Example 3

Amplification of a Pectin Methylesterase Gene From Pear (PcPME)

Pear mesocarp processing, RNA extraction, mRNA isolation and RT reaction were performed exactly as described for β-galactosidase isolation in Example 1.

The cDNA produced was amplified with 2.0 U Taq DNA polymerase (Gibco-BRL) in a 20 mM Tris-HCl pH 8.4 and 50 mM KCl mixture containing 3.0 mM MgCl2, 0.25 mM each dNTP and 20 pmol of each primer PME1 (SEQ. ID. NO: 24) and PME2 (SEQ. ID. NO: 25) (see Table1). After an initial 5 min denaturation period at 94° C., the PCR parameters were 30 sec template denaturation at 94° C., 30 sec primer annealing at 50° C. and 1 min primer extension at 72° C. for 35 cycles. A final extension step of 10 min at 72° C. was used subsequently to ensure full-length amplification products. The termocycler used was a Perkin Elmer—Gene Amp PCR System 2400.

The obtained product was purified from the agarose gel and ligated into the vector pBluescript (KS+) (Stratagene). The ligated mixture was used to transform E. coli DH5α. Transformants were selected on LB agar plates containing ampicilin (100 μg/ml) X-gal (80 μg/ml) and IPTG (0.5 mM). Plasmid DNA was isolated using alkaline lysis method.

DNA sequencing was performed in an automated sequencer ABI 310 Applied Biosystems, using Big Dye Terminator Cycle Sequencing Ready Reaction kit (Applied Biosystems).

The PCR obtained band has approximately 200 bp that corresponds only to 15% of coding region. In order to try to isolate whole gene a 5′ RACE reaction was performed using the Marathon cDNA. Also a new primer was designed: PME3 (an antisense primer for 5′ RACE) (SEQ. ID. NO: 26)

For 5′ RACE reaction, Marathon cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of primers PME3 (SEQ. ID. NO: 26) (see Table 1) and AP1 (provided with Marathon kit, Clontech). After an initial 5 min denaturation period at 94° C., the PCR parameters were 30 sec at 94° C., 30 sec at 50° C. and 1 min at 72° C. for 35 cycles and a final extension step of 10 min at 72° C. The approximately 600 bp PCR product was cloned and sequenced as described above.

Both fragments together comprise a cDNA molecule of 700 bp in size (SEQ. ID. NO: 5) and represents about 60% of the coding region.

The PME nucleotide sequence was sent to NCBI data bank and has shown significant homology with pectin methylesterases isolated from other species. Searches in all the available protein and DNA data banks failed to find 100% homology with any existing clone.

### Example 4

Amplification of Two Expansin Genes From Pear (PcExp1 and PcExp2)

Pear mesocarp processing, RNA extraction, mRNA isolation and RT reaction were performed exactly as described for β-galactosidase isolation in Example 1.

The cDNA produced was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of each degenerated primers EX1 (SEQ. ID. NO: 27) and EX2 (SEQ. ID. NO: 28) (see Table1). After an initial 5 min denaturation period at 94° C., the PCR parameters were 30 sec template denaturation at 94° C., 30 sec primer annealing at 58° C. and 45 sec primer extension at 72° C. for 35 cycles. A final extension step of 10 min at 72° C. was used subsequently to ensure full-length amplification products. The termocycler used was a Perkin Elmer—Gene Amp PCR System 2400.

An approximately 300 bp expected band was obtained. This product was purified from the agarose gel and ligated into the vector pBluescript (KS+) (Stratagene). The ligated mixture was used to transform E. coli DH5α. Transformants were selected on LB agar plates containing ampicilin (100 μg/ml) X-gal (80 μg/ml) and IPTG (0.5 mM). Plasmid DNA was isolated using alkaline lysis method. DNA sequencing was performed in an automated sequencer ABI 310 Applied Biosystems, using Big Dye Terminator Cycle Sequencing kit (Applied Biosystems).

The PCR obtained band of approximately 300 bp corresponds only to 30% of the coding region. In order to isolate whole gene RACE reactions were performed—5′ RACE reaction using the Marathon cDNA and 3′ RACE using cDNA from an RT performed as described in Example 1. Also, new primers were designed: EX3 (SEQ. ID. NO: 29) (an antisense primer for 5′ RACE) and EX4 (SEQ. ID. NO: 30) (a sense primer for 3′ RACE).

For 5′ RACE reaction, Marathon cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of EX3 (SEQ. ID. NO: 29) (see Table 1) and AP1 (Adaptor Primer provided with Marathon kit, Clontech) primers. After an initial 5 min denaturation period at 94° C., the PCR parameters were 30 sec at 94° C., 45 sec at 42 ° C. and 1 min at 72° C. for 35 cycles and a final extension step of 10 min at 72° C. When cloned, the approximately 500 bp PCR product showed two distinct patterns when cut with EcoRI and Hind III restriction enzymes. Both clones were then sequenced and revealed to be different expansin gene fragments. The first one corresponds to 5′ region of the 300 bp Expansin 1 gene isolated. The second one was Expansin 2 5′ end. For the 3′ RACE reaction of Exp1, cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of each EX4 (SEQ. ID. NO: 30) (see Table1) and Vial9 primers (provided with 5′/3′ Race kit, Boehringer). After an initial 5 min denaturation period at 94° C., the PCR parameters were 30 sec at 94° C., 45 sec at 48° C. and 1 min at 72° C. for 35 cycles and a final extension step of 10 min at 72° C. The approximately 700 bp PCR product was cloned and sequenced. For the 3′ RACE reaction of Exp2, cDNA was amplified with 2.0 U Taq DNA polymerase (Gibco-BRL) in a 20 mM Tris-HCl pH 8.4 and 50 mM KCl mixture containing 2.0 mM MgCl2, 0.25 mM each dNTP and 10 pmol of primers EX5 (SEQ. ID. NO: 31) (see Table1) and Vial9 (Boehringer). After an initial 5 min denaturation period at 94° C., the PCR parameters were 30 sec at 94° C., 45 sec at 60° C. and 2 min at 72° C. for 35 cycles and a final extension step of 10 min at 72° C. The approximately 600 bp PCR product was cloned and sequenced.

Exp1 sequence has 1276 bp (SEQ. ID. NO: 7) and Exp2 has 1144 bp (SEQ. ID. NO: 9). These nucleic acid sequences encode two different Expansin proteins and each sequence corresponds to 100% of the respective coding region.

The complete nucleotide sequences of Exp1 and Exp2 were sent to NCBI data bank and have shown significant homology with Expansins isolated from other species. The highest homology found at the DNA level using the blastn program for Exp1 was 86% with about 600 base pairs of Fragaria x ananassa Exp1 mRNA clone # AF 163812, and for Exp2 90% with about 800 base pairs of Prunus cerasus expansin2 mRNA clone #AF350937. Searches in all the available protein and DNA data banks failed to find 100% homology with any existing clone.

The primers used for the first PCR are preferably degenerated primers, which are choosen in conserved portions of different isoforms of the same gene isolated before from other organisms. The other specific primers were designed for 5′ and 3′ RACE using as template the nucleic acid sequences previously obtained by PCR. Table 1 presents all the designed primers used for gene isolation.

TABLE 1

| | | |
|---|---|---|
| BG1: | 5′-TGG(T/C)TC(T/C)ATTCA(T/C)TA(T/C)CC(T/C)AGAAG-3′ | (SEQ. ID. NO:17) |
| BG2: | 5′-CA(C/A/T)GAIC(G/T)(T/A)GGAA(C/T)(A/G)TG(A/G)TACCAT-3′ | (SEQ. ID. NO:18) |
| BG3: | 5′-GCCTCCATCTTTGGCCTTCTGAAT-3′ | (SEQ. ID. NO:19) |
| PG1: | 5′-AG(C/T)CC(C/T)AA(C/T)AC(C/T)GA(C/T)GGIAT(C/T)CA-3′ | (SEQ. ID. NO:20) |
| PG2: | 5′-A(A/G)(A/G)CTICC(A/G)AT(A/G)CT(G/T)ATICC(A/G)TG-3′ | (SEQ. ID. NO:21) |
| PG3: | 5′-AGTCGAGAATGGTGACTCCAGAT-3′ | (SEQ. ID. NO:22) |
| PG4: | 5′-GGCACTACCAATTTGTGGATTGA-3′ | (SEQ. ID. NO:23) |
| PME1: | 5′-ACCGTCGATTTCATTTTCGGA-3′ | (SEQ. ID. NO:24) |
| PME2: | 5′-AAACCATGGCCTACCAAGATA-3′ | (SEQ. ID. NO:25) |
| PME3: | 5′-CCCTGTATTGTAATAGTTGCA-3′ | (SEQ. ID. NO:26) |
| EX1: | 5′-AC(A/G)(A/T)(T/C)GG(T/C)GGITGGTG(T/C)AA(T/C)CC-3′ | (SEQ. ID. NO:27) |
| EX2: | 5′-TGCCA(G/A)TT(G/T)(G/T)(C/G)ICCCA(A/G)TT(C/T)C-3′ | (SEQ. ID. NO:28) |
| EX3: | 5′-CGGTATTGGGCAATTTGCAAGAA-3′ | (SEQ. ID. NO:29) |
| EX4: | 5′-GGATATCGTGAGGGTGAGCGTAA-3′ | (SEQ. ID. NO:30) |
| EX5: | 5′-GGAGACGTCCATTCAGTTTCAAT-3′ | (SEQ. ID. NO:31) |

1. Five isolated nucleic acid sequences from pear fruit comprising encoding regions for β-galactosidase (Pcβ-gal), pectin methylesterase (PcPME), polygalacturonase (PcPG), expansin1 (PcExp1) and expansin2 (PcExp2) proteins.

2. The isolated nucleic acid molecule, according to claim 1, wherein the polynucleotide has the sequence of SEQ. ID. NO: 1.

3. The isolated nucleic acid sequence according to claim 2, wherein the polynucleotide encodes a β-Galactosidase polypeptide.

4. The isolated nucleic acid sequences according to claim 2, wherein the polynucleotide encodes a protein or polypeptide having an aminoacid sequence of SEQ. ID. NO: 2.

5. The isolated nucleic acid molecule, according to claim 1, wherein the polynucleotide has the sequence of SEQ. ID. NO: 3.

6. The isolated nucleic acid sequences according to claim 5, wherein the polynucleotide encodes a Polygalacturonase polypeptide.

7. The isolated nucleic acid sequences according to claim 5, wherein the polynucleotide encodes a protein or polypeptide having an aminoacid sequence of SEQ. ID. NO: 4.

8. The isolated nucleic acid molecule, according to claim 1, wherein the polynucleotide has the sequence of SEQ. ID. NO: 5.

9. The isolated nucleic acid sequences according to claim 8, wherein the polynucleotide encodes a Pectin methylesterase polypeptide.

10. The isolated nucleic acid sequences according to claim 8, wherein the polynucleotide encodes a protein or polypeptide having an aminoacid sequence of SEQ. ID. NO: 6.

11. The isolated nucleic acid molecule, according to claim 1, wherein the polynucleotide has the sequence of SEQ. ID. NO: 7.

12. The isolated nucleic acid sequences according to claim 11, wherein the polynucleotide encodes an Expansin polypeptide said Exp1.

13. The isolated nucleic acid sequences according to claim 11, wherein the polynucleotide encodes a protein or polypeptide having an aminoacid sequence of SEQ. ID. NO: 8.

14. The isolated nucleic acid molecule, according to claim 1, wherein the polynucleotide has the sequence of SEQ. ID. NO: 9.

15. The isolated nucleic acid sequences according to claim 14, wherein the polynucleotide encodes an Expansin polypeptide said Exp2.

16. The isolated nucleic acid sequences according to claim 14, wherein the polynucleotide encodes a protein or polypeptide having an aminoacid sequence of SEQ. ID. NO: 10.

17. The isolated nucleic acid sequences according to claim 1, presented as RNA, mRNA, cRNA, DNA or cDNA molecules.

18. A nucleic acid fragment of at least 30 nucleotide homologous to any of the isolated nucleic acid sequences of claim 1.

19. The isolated nucleic acid sequences described in claim 1, which can be used together with other genes expressed in pear fruit.

20. A chimeric gene comprising one or more nucleic acid molecules according to claim 1 in sense or antisense orientation and which can be operably linked to a promoter.

21. A chimeric gene comprising at least one nucleic acid fragment according to claim 18 in sense or antisense orientation and which can be operably linked to a promoter.

22. Any expression cassette comprising at least one of the chimeric genes described in claim 20 and 21.

23. Any replicable expression vector comprising at least one of the chimeric genes described in claim 20 and 21.

24. A plant genome comprising at least one of the chimeric genes described in claim 20 and 21.

25. A host cell transformed with at least one of the chimeric genes described in claim 20 and 21.

26. A genetically modified plant containing at least one of the chimeric genes described in claim 20 and 21, wherein said chimeric gene is stably integrated into the plant genome.

27. The progeny of cross breeding involving the plant described in claim 26.

28. The fruit or seeds comprising at least one of the chimeric genes described in claim 20 and 21, wherein said chimeric gene is stably integrated into the plant genome.

29. Any method of modifying softness in fruits of a plant, the method comprising introduction into the plant an expression cassette according to the described in claim 22.

30. Any method of modifying cell walls in the tissues of a plant, the method comprising introduction into the plant an expression cassette according to the described in claim 22.

31. Any method of modifying plant cell walls response to physiological processes or biological agents, such as fruit ripening or pathogen attack, the method comprising introduction into the plant an expression cassette according to the described in claim 22.
